(19) United States
(12) Patent Application Publication
(10) Pub. No.: US 2025/0026603 A1
(43) Pub. Date: Jan. 23, 2025

(54) INSPECTION SYSTEM, INSPECTION METHOD, AND NON-TRANSITORY RECORDING MEDIUM STORING COMPUTER-READABLE INSPECTION PROGRAM

(71) Applicant: Konica Minolta, Inc., Tokyo (JP)

(72) Inventor: HIROMU NAGATO, Tokyo (JP)

(21) Appl. No.: 18/763,388

(22) Filed: Jul. 3, 2024

(30) Foreign Application Priority Data
Jul. 18, 2023 (JP) 2023-116564

(51) Int. Cl. B65H 43/04; B65H 29/60; B65H 37/04; G01N 21/88; G01N 33/34; G06T 7/00

(52) U.S. Cl. CPC B65H 43/04; B65H 29/60; B65H 37/04; G01N 21/8851; G06T 7/0004; B65H 2301/5111; B65H 2801/24; G01N 2021/8887; G01N 33/346; G06T 2207/20081; G06T 2207/30144

(57) ABSTRACT

An inspection system can inspect a bundle including a plurality of recording media. The inspection system includes an acquirer that acquires information regarding a shape of a bundle including a plurality of processed recording media, and a hardware processor, in which the hardware processor inspects the bundle based on the information acquired by the acquirer.

### CROSS-REFERENCE TO RELATED APPLICATION

The entire disclosure of Japanese patent Application No. 2023-116564, filed on Jul. 18, 2023, is incorporated herein by reference in its entirety.

### BACKGROUND OF THE INVENTION

### 1. Technical Field

The present invention relates to an inspection system, an inspection method, and a non-transitory recording medium storing a computer-readable inspection program.

### 2. Description of Related Art

Usually, post-processing such as booklet binding is performed after printing. Conventionally, a technique for inspecting a printed product generated by printing is known. For example, Japanese Unexamined Patent Application Publication No. 2017-88322 describes “a system for inspecting both a paper curling defect and a shrinkage defect by using a sensor for detecting the surface height of a printing sheet”.

### SUMMARY OF THE INVENTION

However, conventionally, there has been no technique for inspecting a bundle including a plurality of recording media. Therefore, the work of inspecting a booklet or the like generated by post-processing has been required to be manually performed.

An object of the present invention is to provide an inspection system, an inspection method, and a non-transitory recording medium storing a computer-readable inspection program that are capable of inspecting a bundle including a plurality of recording media.

An inspection system according to the present invention includes an acquirer that acquires information regarding a shape of a bundle including a plurality of processed recording media, and a hardware processor, in which the hardware processor inspects the bundle based on the information acquired by the acquirer.

An inspection method according to the present invention includes acquiring information regarding a shape of a bundle including a plurality of processed recording media, and inspecting the bundle based on the acquired information.

A non-transitory recording medium stores a computer-readable inspection program according to the present invention, the inspection program causing a computer to execute: acquiring information regarding a shape of a bundle including a plurality of processed recording media; and inspecting the bundle based on the acquired information and determining whether or not the bundle is a non-defective product.

### BRIEF DESCRIPTION OF THE DRAWINGS

The advantages and features provided by one or more embodiments of the invention will become more fully understand from the detailed description given hereinbelow and the appended drawings which are given by way of illustration only, and thus are not intended as a definition of the limits of the present invention.

FIG. 1 is a schematic diagram illustrating an example of a configuration of an inspection system according to an embodiment of the present invention;

FIG. 2 is a diagram for explaining an example of saddle stitch binding;

FIG. 3 is a diagram for explaining an example of stapleless binding;

FIG. 4 is a functional block diagram illustrating a configuration of a part of the inspection system according to the embodiment of the present invention;

FIG. 5 is a diagram illustrating an example of a monochrome heat map corresponding to a non-defective bundle of sheets;

FIG. 6 is a diagram illustrating an example of a monochrome heat map corresponding to a defective bundle of sheets;

FIG. 7 is a diagram illustrating an example of two-dimensional contour lines corresponding to a non-defective bundle of sheets;

FIG. 8 is a diagram illustrating an example of two-dimensional contour lines corresponding to a defective bundle of sheets;

FIG. 9 is a schematic diagram illustrating a procedure of non-defective product learning;

FIG. 10 is a schematic diagram illustrating a procedure of inference using a learning model;

FIG. 11 is a diagram illustrating a case where there is no difference between an original image and a generated image;

FIG. 12 is a diagram illustrating a case where there is a difference between an original image and a generated image;

FIG. 13 is a diagram illustrating an example of a bundle of sheets having a defective portion;

FIG. 14 is a flowchart illustrating an example of a method for constructing a learning model;

FIG. 15 is a diagram illustrating normalization performed in a surface direction of a bundle of sheets;

FIG. 16 is a diagram illustrating normalization performed in a height direction of a bundle of sheets;

FIG. 17 is a diagram for explaining an example in which the shape of a bundle of sheets in the height direction changes depending on the basis weight of a cover sheet;

FIG. 18 is a diagram illustrating an example in which the shape of a bundle of sheets in the height direction changes depending on the number of sheets; and

FIG. 19 is a flowchart illustrating an example of a method for inspecting a bundle of sheets using the inspection system according to the embodiment of the present invention.

### DETAILED DESCRIPTION

Hereinafter, embodiments of the present invention will be described in detail with reference to the drawings. In this specification and the drawings, elements having substantially the same functions or configurations are denoted by the same reference numerals, and redundant description thereof will be omitted. The present invention relates to a technique for inspecting a bundle including a plurality of processed recording media. In the following embodiment, a case where the recording media are sheets will be described as an example.

Provided that the recording media are not limited to sheets.

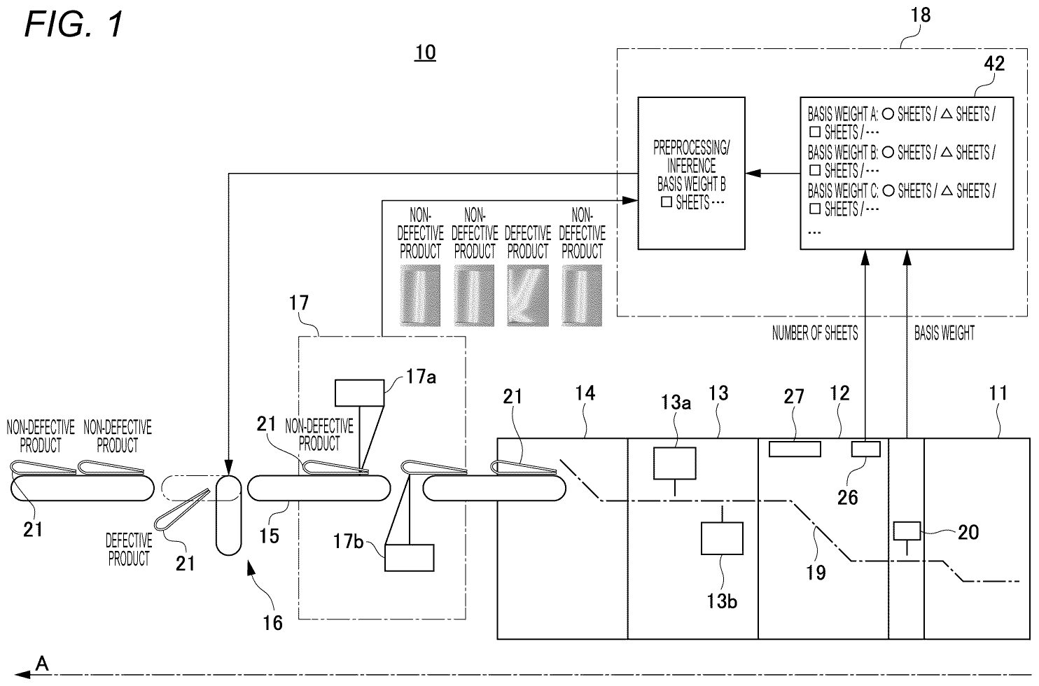

FIG. 1 is a schematic diagram illustrating an example of a configuration of an inspection system according to an embodiment of the present invention.

As illustrated in FIG. 1, the inspection system 10 includes a sheet feeding machine 11, a printing machine 12, an image inspection machine 13, a post-processing machine 14, a bundle conveyance machine 15, an ejection mechanism 16, an acquirer 17, and an inspection computer 18. In the drawing, a leftward arrow A indicates a sheet passing direction.

The sheet feeding machine 11 feeds sheets. The sheet feeding machine 11 feeds sheets stored in a tray or the like one by one. The printing machine 12 prints an image on one side or both sides of a sheet supplied by the sheet feeding machine 11. The printing machine 12 corresponds to a printer.

The image inspection machine 13 inspects the image printed on the sheet by the printing machine 12. The image inspection machine 13 includes a first image inspection sensor 13a that inspects an image printed on a first surface of a sheet and a second image inspection sensor 13b that inspects an image printed on a second surface of the sheet. The sheet is conveyed along a conveyance path 19 from the sheet feeding machine 11 to the post-processing machine 14 by a sheet conveyance mechanism (not illustrated). FIG. 1 does not illustrate a conveyance path for double-sided printing.

A medium sensor 20 is disposed on the way of the conveyance path 19 directed to the printing machine 12 from the sheet feeding machine 11. The medium sensor 20 detects information regarding the sheet. The information detected by the medium sensor 20 includes the basis weight of the sheet. The printing machine 12 includes an operation part 26 and a system controller 27.

The operation part 26 functions as a user interface. Although not illustrated, the operation part 26 includes an input part and a display part. The input part receives an input of various kinds of information from a user. The display part displays various kinds of information to the user. The information received by the input part includes information regarding a print job. The information regarding the print job includes the number of sheets forming a bundle (hereinafter, also referred to as a “bundle of sheets”) including the plurality of sheets. The bundle of sheets is generated by processing of the post-processing machine 14. Instead of the medium sensor 20 that acquires the basis weight of the sheet, for example, the user may input the basis weight of the sheet via the operation part 26.

The system controller 27 comprehensively controls the operation of the entire inspection system 10. Targets to be controlled by the system controller 27 include the sheet feeding machine 11, the printing machine 12, the image inspection machine 13, the post-processing machine 14, the bundle conveyance machine 15, the ejection mechanism 16, the acquirer 17, and the like.

The post-processing machine 14 generates a bundle including a plurality of sheets, that is, a bundle 21 of sheets by the processing. The post-processing machine 14 corresponds to a processing section. The post-processing machine 14 is disposed downstream of the printing machine 12 in the sheet passing direction A. Therefore, a plurality of sheets on which images have been printed by the printing machine 12 are processed by the post-processing machine 14. The post-processing machine 14 is disposed downstream of the image inspection machine 13 in the sheet passing direction A. Therefore, the plurality of sheets on which the images have been inspected by the image inspection machine 13 are processed by the post-processing machine 14. The post-processing machine 14 uses the plurality of sheets on which the images have been printed by the printing machine 12 and have been inspected by the image inspection machine 13 to generate, by processing, a bundle 21 of sheets including the plurality of sheets. Note that a sheet determined to have a printing failure by the image inspection machine 13 may be fed to a sheet ejection section (not illustrated) disposed upstream of the processing section of the post-processing machine 14 and may be removed from targets subject to the processing. Since the sheet having the printing failure is removed before the processing, at least the sheet having the printing failure is not included in the sheets subject to the processing. In a case where the sheet having the printing failure is not removed before the processing, a storage stores results of the inspection by the image inspection machine 13, and the bundle 21 of sheets including the target sheet is sorted as a defective product after the processing in order to distinguish the defective product from a non-defective product. Since the processing is performed after printing, it is also called post-processing.

The bundle of sheets includes at least one of the following (A) to (C).
(A) A bundle of a plurality of stacked sheets without folding
(B) A bundle obtained by folding and stacking a plurality of sheets
(C) A bundle obtained by folding one sheet

The post-processing machine 14 processes the plurality of sheets based on conditions designated in the print job. In general, there are many kinds of processing. For example, the processing include stapling, punching, case binding, folding, saddle stitch binding, stapleless binding, and perfect binding. The stapleless binding is also called scram binding or empty binding. Binding of booklets is one form of the processing. A booklet is obtained by binding the bundle of (B) described above or the bundle of (C) described above as a booklet. Booklets to be bound are roughly classified into booklets that need to be bound and booklets that do not need to be bound. Booklets that need to be bound include a saddle-stitched booklet. Booklets that do not need to be bound include an unbound booklet. Furthermore, as an example of a bundle of sheets that is not a booklet, there is a bundle of sheets obtained by performing stapling or punching on the bundle of sheets of (A) described above.

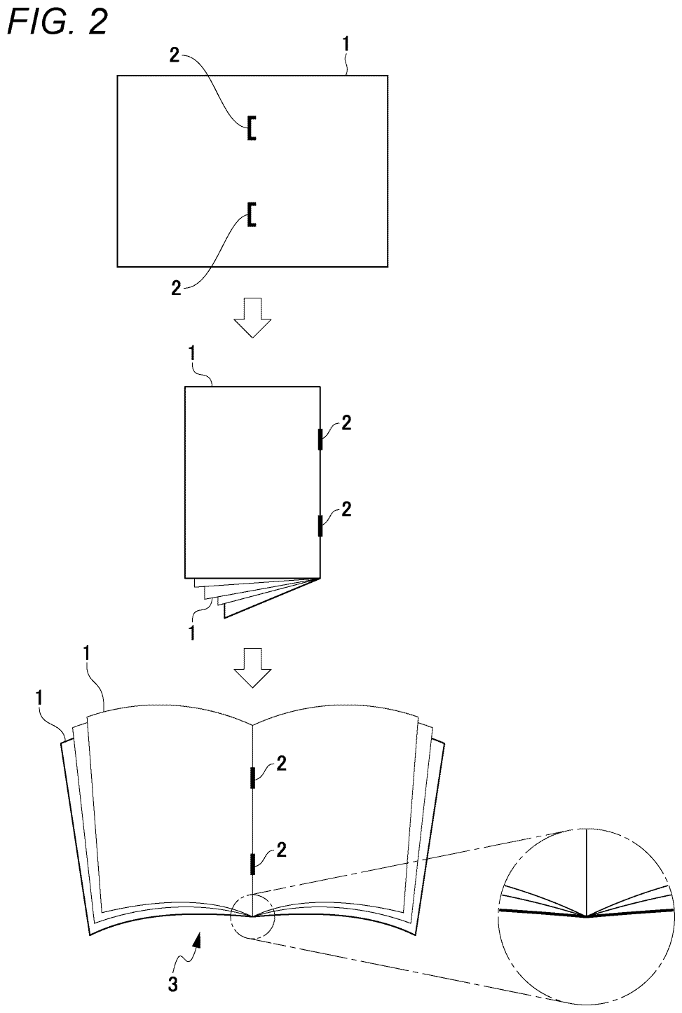

FIG. 2 is a diagram for explaining an example of saddle stitch binding.

As illustrated in FIG. 2, in the saddle stitch binding, first, in a state where a plurality of sheets 1 are stacked, a plurality of staples 2 are driven on the center lines of the sheets 1. Next, the plurality of sheets 1 are folded along the center lines. Thus, a saddle-stitched booklet, that is, a saddle-stitched booklet 3 is obtained.

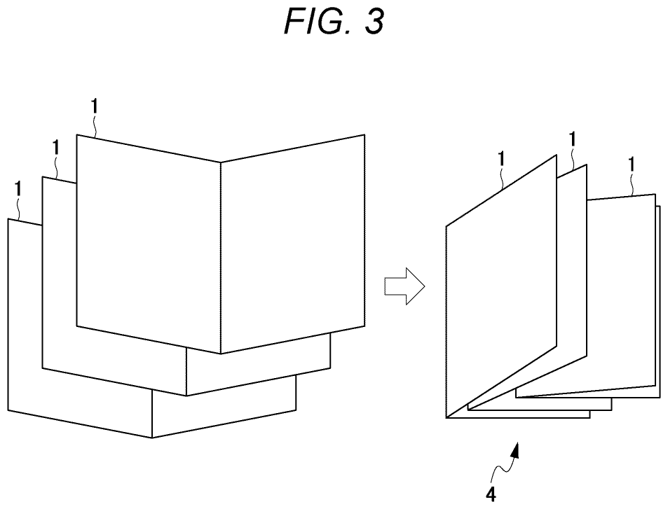

FIG. 3 is a diagram for explaining an example of stapleless binding.

As illustrated in FIG. 3, in the stapleless binding, a plurality of sheets 1 folded in two are put together in the form of a booklet by being stacked one on another in the order of pages. As a result, a booklet obtained by stapleless binding, that is, an unbound booklet 4 is obtained. The booklet subjected to the stapleless binding is a booklet that is not bound with a staple, a thread, or the like, and therefore is also called an empty bound booklet or the like.

In the unbound booklet 4, defects such as a fold and a tear of a sheet 1 may occur. Besides defects such as the fold and the tear of the sheet 1, a defect related to the stapling may occur in the saddle-stitched booklet 3. The defect related to the stapling includes, for example, idle driving of a staple 2 and buckling of a staple 2. Positions at which the staples 2 are to be driven are designated as one of the conditions for the processing described above. Therefore, it is possible to identify a portion where the defect related to the stapling occurs. On the other hand, a portion where a defect such as a fold or a tear of a sheet 1 occurs is not determined in many cases. In addition, in many cases, it is not determined in which shape the sheet 1 is bent or torn. Therefore, it is difficult to identify a portion where the defect such as the fold or the tear of the sheet 1 occurs.

The bundle conveyance machine 15 conveys the bundle including the plurality of sheets processed by the post-processing machine 14, that is, the bundle 21 of sheets. The bundle conveyance machine 15 includes, for example, a plurality of belt conveyors.

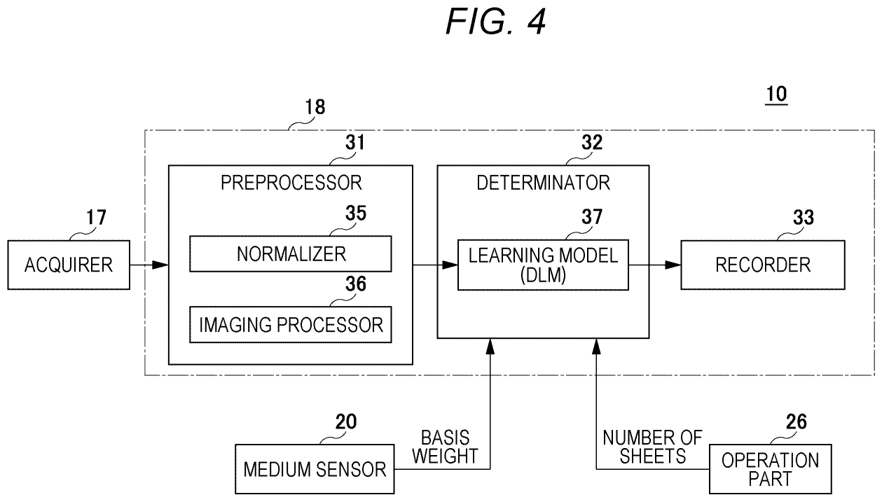

The ejection mechanism 16 switches the conveyance destination of the bundle of sheets in accordance with a result of inspection by a determinator 32 (FIG. 4). The ejection mechanism 16 corresponds to an ejector. The determinator 32 determines whether or not the bundle of sheets to be inspected is a non-defective product. Details of the determinator 32 will be described later. The ejection mechanism 16 has a posture changing function. The posture changing function is a function of changing the posture of the ejection mechanism 16 between a first posture and a second posture. The first posture is a posture indicated by a two-dot chain line in FIG. 1. The second posture is a posture indicated by a solid line in FIG. 1.

When the ejection mechanism 16 is set to the first posture, the conveyance destination of the bundle of sheets is switched to a non-defective product collecting section (not illustrated). When the ejection mechanism 16 is set to the second posture, the conveyance destination of the bundle of sheets is switched to a defective product collecting section (not illustrated). When the determinator 32 determines that the bundle of sheets to be inspected is a non-defective product, the ejection mechanism 16 is maintained in the first posture such that the conveyance destination of the non-defective bundle of sheets is the non-defective product collecting section.

When the determinator 32 determines that the bundle of sheets to be inspected is not a non-defective product, that is, is a defective product, the ejection mechanism 16 changes from the first posture to the second posture such that the conveyance destination of the defective bundle of sheets is the defective product collecting section.

The acquirer 17 acquires information regarding the shape of the bundle including the plurality of sheets subjected to the processing.

The above-described information acquired by the acquirer 17 is preferably three-dimensional information. The three-dimensional information includes information regarding the shape of the bundle of sheets in a surface direction and information regarding the shape of the bundle of sheets in a height direction. The shape of the bundle of sheets in the surface direction is a shape when the bundle of sheets is viewed in plan view, in other words, a shape defining the size of the bundle of sheets. The height direction of the bundle of sheets can be rephrased as a thickness direction of the bundle of sheets. Furthermore, the three-dimensional information can also be rephrased as information indicating the three-dimensional shape of the bundle of sheets.

The acquirer 17 includes a first acquirer 17a and a second acquirer 17b. The first acquirer 17a acquires information regarding the shape of the bundle of sheets on the front side of the bundle of sheets. The second acquirer 17b acquires information regarding the shape of the bundle of sheets on the back side of the bundle of sheets. The first acquirer 17a is disposed above the bundle conveyance machine 15. When the first acquirer 17a is disposed in this way, the front side of the bundle of sheets corresponds to the upper side of the bundle 21 of sheets conveyed by the bundle conveyance machine 15. The second acquirer 17b is disposed below the bundle conveyance machine 15. When the second acquirer 17b is arranged in this way, the back side of the bundle of sheets corresponds to the lower side of the bundle 21 of sheets conveyed by the bundle conveyance machine 15. In the following description, the first acquirer 17a and the second acquirer 17b are simply referred to as the acquirer 17 when there is no need to distinguish them.

The acquirer 17 acquires information regarding the shape of the bundle 21 of sheets by using light emitted to the bundle 21 of sheets. The acquirer 17 includes, for example, a laser displacement meter of a light cutting method.

The acquirer 17 that includes the laser displacement meter of the light cutting method irradiates the surface of the bundle 21 of sheets with band-like laser light and receives light reflected from the bundle 21 of sheets, thereby acquiring three-dimensional information (three-dimensional data) regarding the shape of the bundle 21 of sheets. In a case where the acquirer 17 includes the laser displacement meter of the light cutting method, the above-described three-dimensional information can be acquired with high accuracy without deforming the shape of the bundle 21 of sheets.

In the present embodiment, as an example of the acquirer 17 that acquires the above-described three-dimensional information using light, the laser displacement meter of the light cutting method has been exemplified, but the present invention is not limited to the laser displacement meter of the light cutting method. For example, the acquirer 17 may be configured to acquire the above-described three-dimensional information by a time of flight (ToF) sensor, a multiple-lens camera, a pattern illumination method, a structured lighting method, or the like. Furthermore, the acquirer 17 may be configured to acquire information regarding the shape of the bundle 21 of sheets by using a sound emitted to the bundle 21 of sheets. Specifically, the acquirer 17 transmits a sound wave toward the surface of the bundle 21 of sheets and receives the sound wave reflected off the surface of the bundle 21 of sheets, thereby acquiring the three-dimensional information regarding the shape of the bundle 21 of sheets. Since the above-described methods using light or sound are all non-contact methods, the above-described three-dimensional information can be acquired without deforming the shape of the bundle 21 of sheets. Furthermore, the acquirer 17 may be configured to acquire the information regarding the shape of the bundle 21 of sheets by using an abutting member that contacts the bundle 21 of sheets. The abutting member is, for example, a probe. In a case where the contact member is used, the contact member comes into contact with the bundle 21 of sheets with a contact pressure that does not deform the shape of the bundle 21 of sheets.

The inspection computer 18 is a computer used to inspect a bundle of sheets. The inspection computer 18 includes a processor such as a graphics processing unit (GPU) or a central processing unit (CPU), and a storage such as a read only memory (ROM), a random access memory (RAM), a hard disk drive (HDD), or a solid state drive (SSD). The inspection computer 18 corresponds to a “hardware processor” according to the present invention.

Each function implemented by the inspection computer 18 will be described later.

FIG. 4 is a functional block diagram illustrating a configuration of a part of the inspection system according to the embodiment of the present invention.

As illustrated in FIG. 4, the inspection system 10 includes the above-described acquirer 17, the inspection computer 18, the medium sensor 20, and the operation part 26. The inspection computer 18 includes a preprocessor 31, the determinator 32, and a recorder 33. The preprocessor 31 takes in and pre-processes the three-dimensional information acquired by the acquirer 17. The preprocessor 31 transmits information (data) generated by the preprocessing to the determinator 32. The preprocessor 31 includes a normalizer 35 and an imaging processor 36. The determinator 32 includes a learning model 37. The information preprocessed by the preprocessor 31 is input to the learning model 37 of the determinator 32.

The normalizer 35 normalizes the three-dimensional information acquired by the acquirer 17 in accordance with the thickness and the size of the bundle of sheets. The normalization is performed as one type of preprocessing. The normalization refers to processing of converting a value to be normalized into a certain range based on a certain rule. As described above, the three-dimensional information acquired by the acquirer 17 includes information regarding the shape of the bundle of sheets in the surface direction and information regarding the shape of the bundle of sheets in the height direction. Furthermore, the information regarding the shape of the bundle of sheets in the surface direction includes numerical values representing the vertical and horizontal sizes of the bundle of sheets. The vertical and horizontal sizes of the bundle of sheets can be rephrased as the lengths of the long side and the short side of the bundle of sheets. On the other hand, the information regarding the shape of the bundle of sheets in the height direction includes a numerical value representing the thickness of the bundle of sheets. The thickness of the bundle of sheets can be rephrased as the height of the bundle of sheets.

The imaging processor 36 generates an image of the three-dimensional information acquired by the acquirer 17. The image generation is performed as one type of preprocessing. The image generation is processing for enabling the three-dimensional information acquired by the acquirer 17 to be handled as an image. Therefore, the image generation is also processing of converting the three-dimensional information into two-dimensional information, that is, two-dimensionalization. Note that the image generated by the image generation of the three-dimensional information includes information regarding the shape of the bundle of sheets in the height direction. Specific examples of the image generated by the image generation include a two-dimensional heat map and two-dimensional contour lines. That is, the three-dimensional information is represented by the two-dimensional heat map or the two-dimensional contour lines.

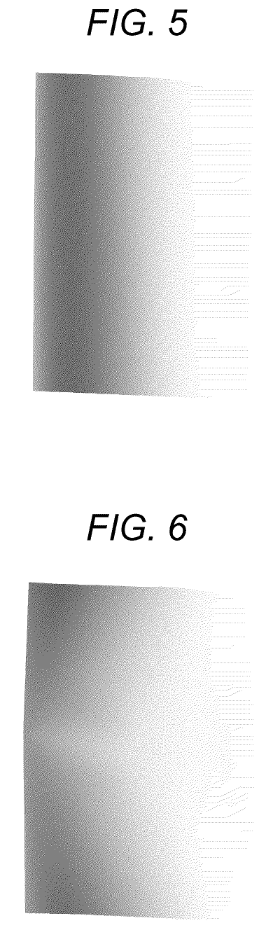

The two-dimensional heat map is divided into two map pieces. One of the two map pieces is an image in which a difference in height is represented by a difference in color. In this case, the two-dimensional heat map is a heat map image having a three-layer structure of red (R), green (G), and blue (B). In the present embodiment, as an example, three-dimensional information from which an image is generated by the imaging processor 36 is represented by a heat map image having the above-described three-layer structure. The other of the map pieces is an image in which a difference in height is represented by a difference in brightness, that is, a single-color gradation. In this case, the two-dimensional heat map is a heat map image having a single-layer structure. The two-dimensional heat map represented by the single-color gradation is, for example, a monochrome heat map. FIG. 5 is a diagram illustrating an example of a monochrome heat map corresponding to a non-defective bundle of sheets. FIG. 6 is a diagram illustrating an example of a monochrome heat map corresponding to a defective bundle of sheets.

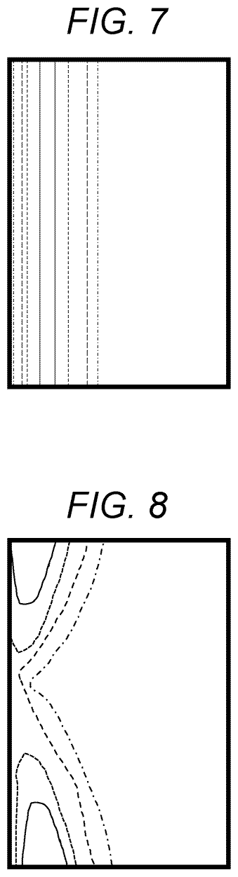

The two-dimensional contour lines are an image in which a difference in height is expressed by a difference in the density of the contour lines. The density of the contour lines increases as an interval between the contour lines decreases. FIG. 7 is a diagram illustrating an example of two-dimensional contour lines corresponding to a non-defective bundle of sheets. FIG. 8 is a diagram illustrating an example of two-dimensional contour lines corresponding to a defective bundle of sheets of.

Note that the image generated by the image generation of the three-dimensional information may be an image in which the two-dimensional heat map and the two-dimensional contour lines are combined.

The determinator 32 inspects the bundle of sheets based on the three-dimensional information acquired by the acquirer 17. The acquirer 17 is disposed downstream of the post-processing machine 14 in the sheet passing direction A. Therefore, the determinator 32 inspects the bundle of sheets processed by the post-processing machine 14. Furthermore, the determinator 32 inspects the bundle of sheets by using the learning model 37. In the inspection of the bundle of sheets performed by the determinator 32, the determinator 32 determines whether or not the bundle of sheets to be inspected is a non-defective product. The determinator 32 determines whether or not the bundle of sheets is a non-defective product by the inference using the learning model 37. As described above, in a case where the acquirer 17 includes the first acquirer 17a and the second acquirer 17b, the determinator 32 can inspect both the front side and the back side of the bundle of sheets. A method for the determination by the determinator 32 will be described later.

The learning model 37 is a deep learning model (deep learning module (DLM)). The deep learning model is a learning model created by deep learning. The use of the deep learning model for inspection of a bundle of sheets enables detection of a defective bundle of sheets as in the case of inspection by a human being. Data normalized by the normalizer 35 is used for the deep learning model. Data of an image generated by the imaging processor 36 is used for the deep learning model.

In the present embodiment, a model of an unsupervised learning method is used as the deep learning model described above. In the case of using the model of the unsupervised learning method, it is not necessary to define a defective product in inspection of a bundle of sheets. For this reason, it is possible to detect a defective bundle of sheets which is not assumed in advance.

In the present embodiment, an autoencoder is used as the deep learning model. The autoencoder belongs to a model of an unsupervised learning method. The autoencoder is a model that is trained such that input data and output data are the same. In the present embodiment, a convolutional autoencoder is used as the deep learning model. The convolutional autoencoder is an autoencoder using a convolutional neural network. The convolutional autoencoder is an autoencoder suitable for learning of an image.

When the determinator 32 determines that the bundle of sheets to be inspected is a defective product, the recorder 33 records information regarding the defective product. The information regarding the defective product includes, for example, information such as the time when the defective product is generated and a defective portion in the defective product. Every time the determinator 32 detects a defective product, information (data) regarding the defective product is accumulated in the recorder 33. The data accumulated in the recorder 33 can be utilized for, for example, machine learning.

Subsequently, a method for constructing the learning model 37 to be used for inspection of a bundle of sheets will be described.

The learning model 37 may be constructed by using the inspection system 10 or a system different from the inspection system 10. In the present embodiment, as a preferred example, the learning model 37 is constructed using the inspection system 10.

First, a worker prepares a number of bundles 21 of sheets necessary for deep learning. The prepared bundles 21 of sheets are all non-defective bundles 21a of sheets. In the present embodiment, the learning model 37 is constructed by unsupervised learning using only the non-defective bundles 21a of sheets, that is, non-defective product learning.

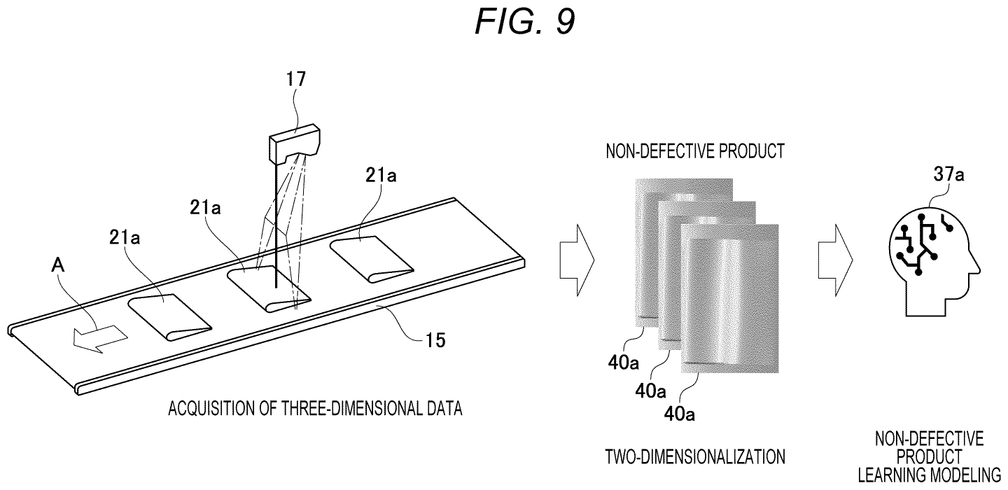

FIG. 9 is a schematic diagram illustrating a procedure of the non-defective product learning.

As illustrated in FIG. 9, the non-defective bundles 21a of sheets are placed on the bundle conveyance machine 15 at intervals in the sheet passing direction A. The non-defective bundles 21a of sheets are conveyed in the sheet passing direction A by the driving of the bundle conveyance machine 15. In this case, the acquirer 17 sequentially acquires three-dimensional information (three-dimensional data) regarding the shape of each of the bundles 21a of sheets.

The imaging processor 36 converts the three-dimensional information acquired by the acquirer 17 into a two-dimensional heat map 40a by generating an image of the three-dimensional information acquired by the acquirer 17 (two-dimensionalization). The converted two-dimensional heat map 40a is input to an AI model 37a. The AI model 37a is a model that functions as image artificial intelligence (AI). The AI model 37a is also a model before learning or during learning. The AI model 37a is trained so as to restore the same image as the input image.

In the present embodiment, a convolutional autoencoder is used as the AI model 37a.

The convolutional autoencoder includes an encoder that converts input data into low-dimensional data by dimensional compression, and a decoder that restores an image from the low-dimensional data. Thus, the AI model 37a is trained so as to restore the same image as the original image with a small amount of information. Therefore, an image output from the AI model 37a using the convolution autoencoder is an image in which only main features of the original image are restored. The trained AI model 37a is used as the learning model 37 for inspecting a bundle of sheets.

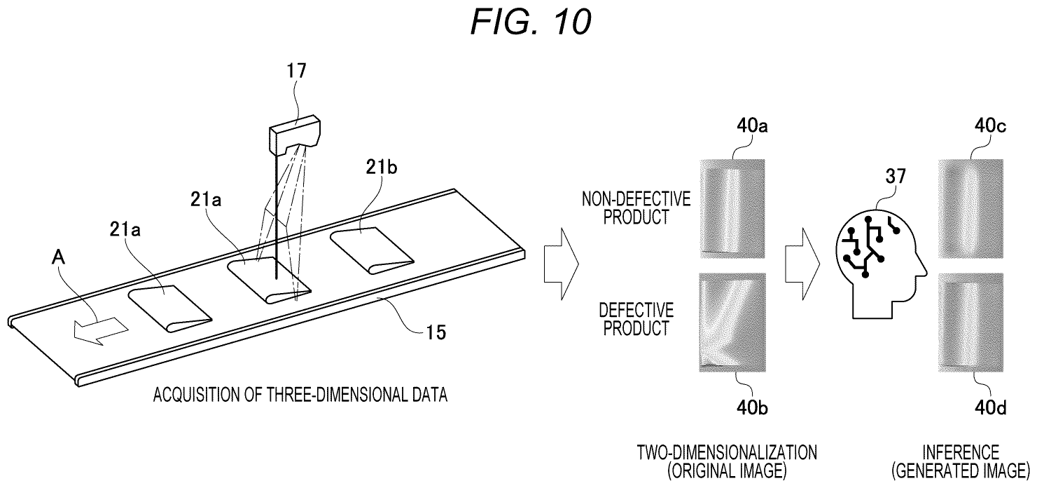

FIG. 10 is a schematic diagram illustrating a procedure of inference using the learning model 37.

As illustrated in FIG. 10, a plurality of bundles 21 of sheets conveyed by the bundle conveyance machine 15 may include a defective bundle 21b of sheets in addition to a non-defective bundle 21a of sheets. In this case, when three-dimensional information of each of the bundles 21a and 21b of sheets is sequentially acquired by the acquirer 17 and images of the acquired three-dimensional information are generated by the imaging processor 36 (two-dimensionalization), different two-dimensional heat maps 40a and 40b are obtained. The two-dimensional heat map 40a is a heat map obtained by generating the image of the three-dimensional information regarding the non-defective bundle 21a of sheets. The two-dimensional heat map 40b is a heat map obtained by generating of the image of the three-dimensional information regarding the defective bundle 21b of sheets.

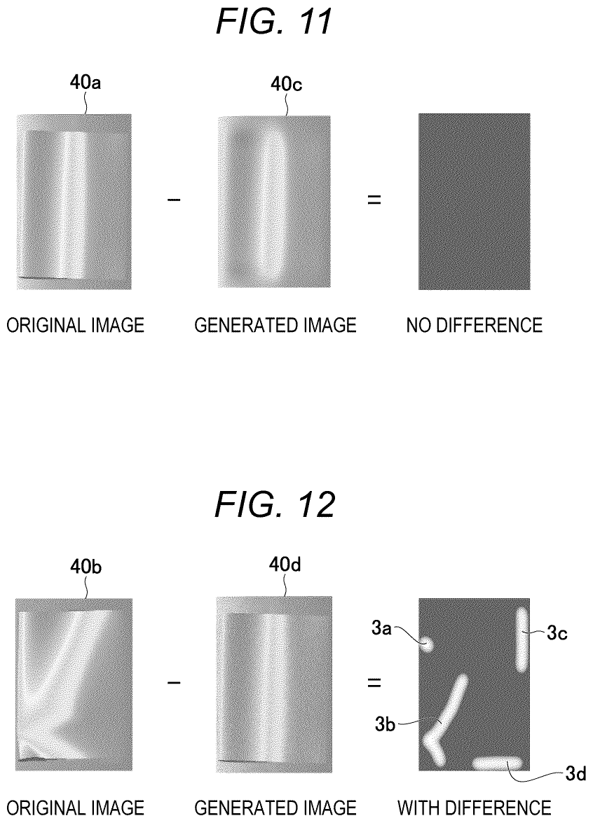

When the two-dimensional heat map 40a is input to the learning model 37, the learning model 37 generates an image 40c by inference based on the non-defective product learning. In the non-defective product learning, the learning model 37 performs learning by using only the non-defective bundle 21a of sheets. Therefore, the image 40c generated by the learning model 37 is substantially the same image as the two-dimensional heat map 40a which is the original image. Therefore, as illustrated in FIG. 11, the difference between the original image 40a input to the learning model 37 and the generated image 40c generated by the learning model 37 is substantially zero. In a case where there is no difference between the original image 40a and the generated image 40c, the determinator 32 determines that the bundle 21a of sheets to be inspected is a non-defective product.

On the other hand, when the two-dimensional heat map 40b is input to the learning model 37, the learning model 37 generates an image 40d by the inference based on the above-described non-defective product learning. In this case, the learning model 37 attempts to restore the original image in accordance with features extracted by the non-defective product learning. However, in the non-defective product learning, the learning model 37 does not learn the information of the defective product. For this reason, the learning model 37 cannot restore a defective portion present in the defective bundle 21b of sheets. Therefore, the image 40d generated by the learning model 37 is substantially different from the two-dimensional heat map 40b that is the original image. Therefore, as illustrated in FIG. 12, the difference between the original image 40b and the generated image 40d does not become zero. In a case where there is a difference between the original image 40b and the generated image 40d as described above, the determinator 32 determines that the bundle 21b of sheets to be inspected is a defective product.

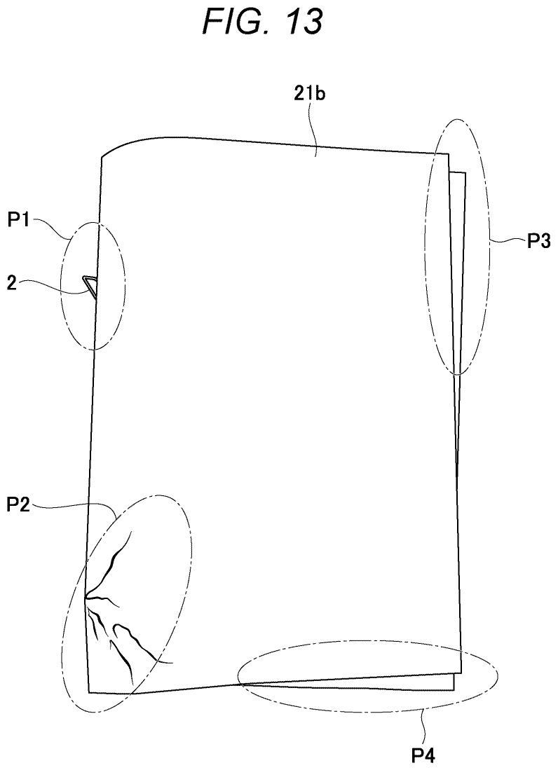

Here, as illustrated in FIG. 13, a case where four defective portions P1, P2, P3, and P4 are present in the bundle 21b of sheets is considered. At the defective portion P1, a staple 2 is not normally driven and buckles, and a part of the staple 2 protrudes from the edge of the bundle 21b of sheets. At the defective portion P2, a fold occurs in the bundle 21b of sheets. When a fold occurs in the bundle 21b of sheets, the height of the folded portion is lower than that of the non-defective bundle 21a of sheets in which no fold occurs. At both of the defective portion P3 and the defective portion P4, edges of the bundle 21 of sheets overlap each other irregularly. In this case, the learning model 37 cannot restore each of the defective locations P1, P2, P3, and P4. Therefore, as illustrated in FIG. 12, the difference between the original image 40b and the generated image 40d includes abnormal feature portions 3a, 3b, 3c, and 3d. The abnormal feature portion 3a appears at the position of the defective portion P1 described above. The abnormal feature portion 3b appears at the position of the defective portion P2 described above. The abnormal feature portion 3c appears at the position of the defective portion P3 described above. The abnormal feature portion 3d appears at the position of the defective portion P4 described above. Thus, the determinator 32 can detect a defective product based on the presence or absence of the above-described difference, and can also identify a defective portion in the detected defective product.

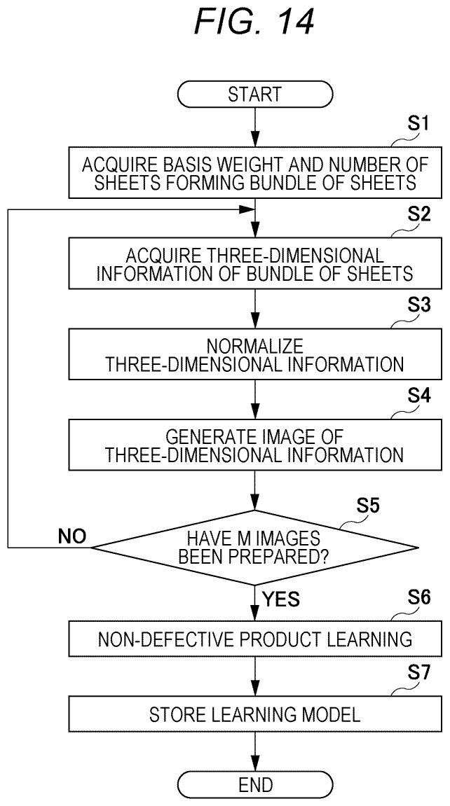

FIG. 14 is a flowchart illustrating an example of a method for constructing the learning model 37.

First, the inspection computer 18 acquires the basis weight of a cover sheet of a bundle of sheets and the number of sheets forming the bundle of sheets as information regarding the bundle of sheets to be used for the above-described non-defective product learning (step S1). The cover sheet of the bundle of sheets is made of paper, but the cover sheet of the bundle including the plurality of recording media may not be necessarily made of paper. Information regarding the basis weight and the number of sheets may be input by the user via the operation part 26. In the present embodiment, the number of bundles of sheets to be used for the non-defective product learning is M. The basis weight and the number of sheets described above are common for the M bundles of sheets used for the non-defective product learning.

Next, the acquirer 17 acquires three-dimensional information regarding one of the M bundles of sheets to be used for the above-described non-defective product learning (step S2). The number of bundles of sheets to be used for the non-defective product learning is determined in accordance with the number of bundles of sheets required for learning of a model by deep learning.

Next, the normalizer 35 normalizes the three-dimensional information acquired by the acquirer 17 in step S2 (step S3).

Here, a specific example of the normalization will be described.

The three-dimensional information to be normalized is information regarding the shape of the bundle of sheets. The three-dimensional information includes information regarding the shape of the bundle of sheets in the surface direction and information regarding the shape of the bundle of sheets in the height direction. Therefore, the normalization of the three-dimensional information is performed in both the surface direction of the bundle of sheets and the height direction of the bundle of sheets.

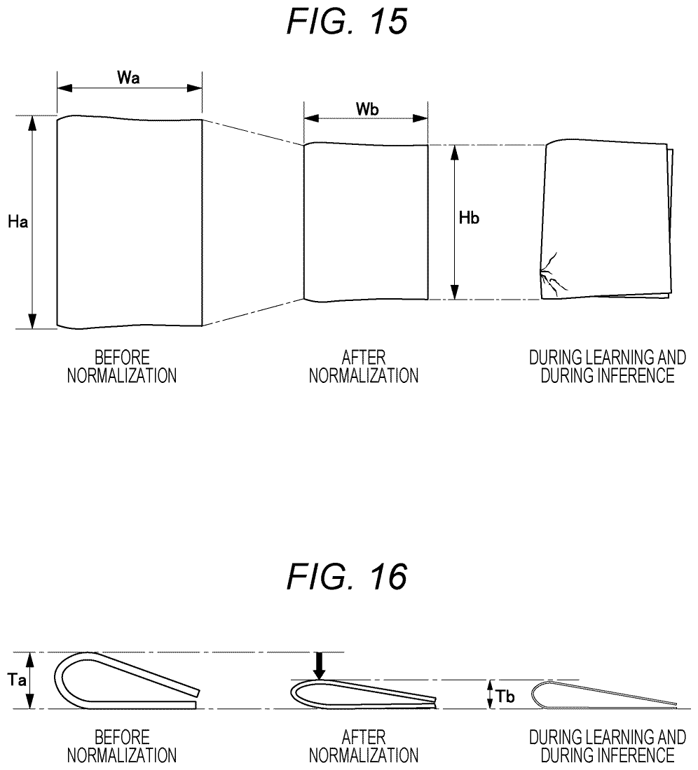

FIG. 15 is a diagram illustrating normalization performed in a surface direction of a bundle of sheets. Note that FIG. 15 illustrates the shape of the bundle of sheets in the surface direction as a two-dimensional image for convenience of explanation.

As illustrated in FIG. 15, the size (Ha×Wa) of the bundle of sheets before being normalized is converted into a size (Hb×Wb) in a predetermined fixed range by being normalized in the surface direction. During the learning illustrated in FIG. 9, the two-dimensional heat map 40a converted into the size (Hb×Wb) in the fixed range by normalizing in the surface direction is input to the AI model 37a. Further, also during the inference illustrated in FIG. 10, the two-dimensional heat maps 40a and 40b converted into the size (Hb×Wb) in the fixed range by normalizing in the surface direction are input to the learning model 37. Thus, the size of the two-dimensional heat map during the learning and the sizes of the two-dimensional heat maps during the inference are made the same.

FIG. 16 is a diagram illustrating normalization performed in a height direction of a bundle of sheets. Note that FIG. 16 illustrates the shape of the bundle of sheets in the height direction as a two-dimensional image for convenience of explanation.

As illustrated in FIG. 16, the height dimension (thickness dimension) Ta of the bundle of sheets before normalization is converted into a height dimension Tb in a predetermined fixed range by normalization in the height direction. Then, the two-dimensional heat map 40a that has been converted into the height dimension Tb in the fixed range by the normalization in the height direction during the learning illustrated in FIG. 9 described above is input to the AI model 37a. Also during the inference illustrated in FIG. 10 described above, the two-dimensional heat maps 40a and 40b normalized in the height direction and converted into the height dimensions Tb in the fixed range are input to the learning model 37. Thus, the height (thickness) of the two-dimensional heat map during the learning and the heights (thicknesses) of the two-dimensional heat maps during the inference are made the same.

By normalizing the three-dimensional information in this way, bundles of sheets having different sizes and bundles of sheets having different thicknesses can be inspected using the same learning model. In a case where the normalization is performed in the height direction of a bundle of sheets, the normalizer 35 may normalize the entire bundle of sheets at a uniform magnification, or may normalize the bundle of sheets by using different magnifications for upper and lower portions of the bundle of sheets.

Returning to FIG. 14 again, a method for constructing the learning model 37 will be described.

Next, the imaging processor 36 generates an image of the three-dimensional information normalized in the above-described step S3 (step S4). In step S4, the imaging processor 36 converts the three-dimensional information into a two-dimensional bitmap by generating the image of the three-dimensional information.

Next, the inspection computer 18 determines whether M two-dimensional bitmaps that are images for learning have been prepared (step S5). In a case where the inspection computer 18 makes a negative determination in step S5, the process returns from step S5 to step S2. The case where the inspection computer 18 makes the negative determination in step S5 is, in other words, a case where the preparation of the images for learning is not completed. In a case where the inspection computer 18 makes an affirmative determination in step S5, the process proceeds from step S5 to step S6. The case where the inspection computer 18 makes the affirmative determination in step S5 is, in other words, a case where the preparation of the images for learning image has been completed.

In step S6, the inspection computer 18 trains the AI model 37a illustrated in FIG. 9 described above using the M images (two-dimensional heat maps) on a non-defective product. To be specific, the inspection computer 18 inputs each of the M two-dimensional heat maps as an input image to the AI model 37a, and trains the AI model 37a so as to output the same images as the input images for all the two-dimensional heat maps.

Next, the inspection computer 18 stores the trained AI model 37a as the learning model 37 in a learning database 42 (see FIG. 1) (step S7). Thus, a model trained with a combination of the above-described basis weight and the above-described number of sheets is stored in the learning database 42. The learning database 42 is one of databases included in the inspection computer 18.

The process procedure illustrated in FIG. 14 is performed for each combination of the basis weight and the number of sheets. Thus, as illustrated in FIG. 1 described above, a model trained for each combination of the above-described basis weight and the above-described number of sheets is stored in the learning database 42.

The reason why the model trained for each combination of the basis weight of the cover sheet of the bundle of sheets and the number of sheets forming the bundle of sheets is stored in the learning database 42 is as follows.

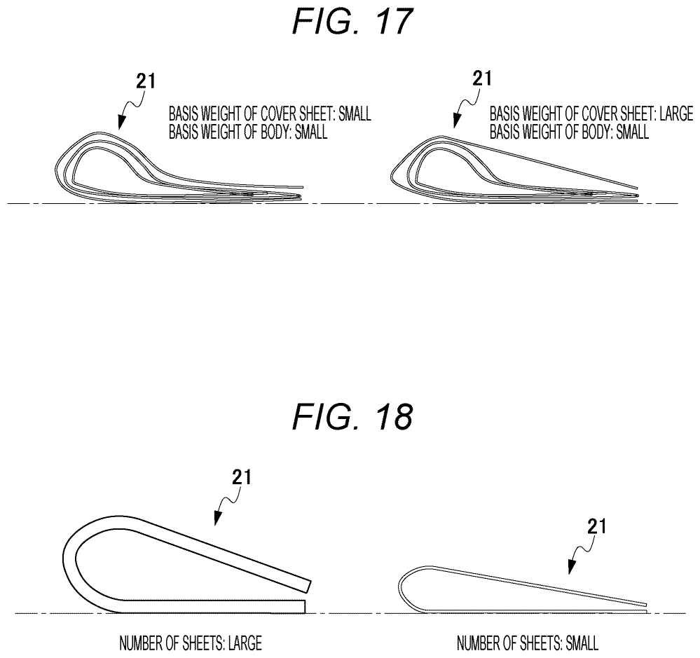

First, a bundle of sheets such as a booklet in which a plurality of sheets are folded and stacked one on another is conceivable. The shape of the bundle of sheets in the height direction is greatly affected by the basis weight of a cover sheet of the bundle of sheets. For this reason, as illustrated in FIG. 17, the shape in the height direction of a bundle 21 of sheets in which both the basis weight of a cover sheet and the basis weight of a body are small is different from the shape in the height direction of a bundle 21 of sheets in which the basis weight of a cover sheet is large and the basis weight of a body is small. Furthermore, a recording medium whose height position is observed by the acquirer 17 in the height direction of a bundle of sheets is the outermost recording medium of the bundle 21 of sheets, that is, a cover sheet of the bundle 21 of sheets. Furthermore, the shape of the bundle of sheets in the height direction is greatly affected by the number of sheets forming the bundle of sheets. Therefore, as illustrated in FIG. 18, the shape of a bundle 21 of a large number of sheets in the height direction is different from the shape of a bundle 21 of a small number of sheets in the height direction.

As described above, in a case where the determinator 32 inspects a bundle of sheets by using the learning model 37, it is preferable to refer to a model corresponding to a combination of the basis weight of a cover sheet of the bundle of sheets and the number of sheets forming the bundle of sheets. Therefore, in the present embodiment, a model trained for each combination of the basis weight of a cover sheet of a bundle of sheets and the number of sheets forming the bundle of sheets is stored in the learning database 42, so that the determinator 32 can read the model to be referred to from the learning database 42.

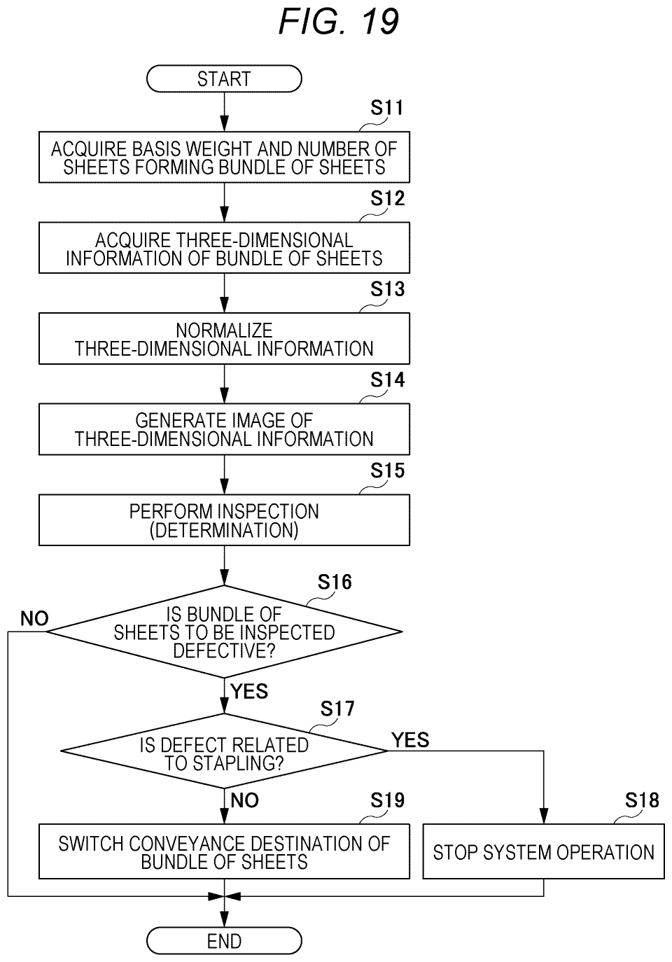

FIG. 19 is a flowchart illustrating an example of a method for inspecting a bundle of sheets using the inspection system according to the embodiment of the present invention. A process in the flowchart illustrated in FIG. 19 is performed for each bundle of sheets.

First, the inspection computer 18 acquires the basis weight of a cover sheet of a bundle of sheets and the number of sheets forming the bundle of sheets as information regarding the bundle of sheets to be inspected (step S11). The basis weight of the cover sheet of the bundle of sheets can be acquired from the medium sensor 20. The number of sheets forming the bundle of sheets can be acquired from the operation part 26.

Next, the acquirer 17 acquires three-dimensional information of the bundle of sheets to be inspected (step S12). In this case, the bundle of sheets for which the acquirer 17 acquires the three-dimensional information is a bundle of sheets processed by the post-processing machine 14.

Next, the normalizer 35 normalizes the three-dimensional information acquired by the acquirer 17 in step S12 (step S13). The normalization of the three-dimensional information is as described above with reference to FIG. 15 and FIG. 16.

Next, the imaging processor 36 generates an image of the three-dimensional information normalized in the above-described step S13 (step S14). In step S14, the imaging processor 36 converts the three-dimensional information into a two-dimensional bitmap by generating the image of the three-dimensional information.

Next, the determinator 32 inspects the bundle of sheets by using the learning model 37 (step S15). In the inspection of the bundle of sheets, the determinator 32 first reads the learning model 37 corresponding to the combination of the basis weight and the number of sheets acquired in step S11 from the learning database 42. Next, the determinator 32 inputs the two-dimensional heat map, which is an image for inspection, to the learning model 37. Thus, the learning model 37 generates an image by inference based on the above-described non-defective product learning. Next, the determinator 32 obtains a difference between the original image and the generated image. Then, in a case where there is no difference between the original image and the generated image (see FIG. 11), the determinator 32 determines that the bundle of sheets to be inspected is a non-defective product. In a case where there is a difference (see FIG. 12) between the original image and the generated image, the determinator 32 determines that the bundle of sheets to be inspected is a defective product. Further, in a case where the bundle of sheets is determined as a defective product, and at least one defective portion is at a position where a staple 2 is driven or in the vicinity of the position, the determinator 32 determines that the defective portion is a defect related to stapling.

Next, the system controller 27 determines, based on the result of the determination by the determinator 32 in the above step S15, whether or not the bundle of sheets to be inspected is a defective product (step S16). If the system controller 27 makes an affirmative determination in step S16, the process proceeds to step S17.

In step S17, the system controller 27 determines, based on the result of the determination by the determinator 32 in step S15, whether or not the defect is related to the stapling. To be specific, when the determinator 32 detects the defect related to the stapling in the above-described step S15, the system controller 27 stops the operation of the system (step S18). Thus, the operations of the sheet feeding machine 11, the printing machine 12, the image inspection machine 13, the post-processing machine 14, the bundle conveyance machine 15, and the like included in the inspection system 10 are stopped. Once a defect related to stapling occurs, a defect tends to occur continuously. Therefore, it is preferable that the system operation be stopped for maintenance of the post-processing machine 14 in a case where the determinator 32 detects a defect related to stapling.

When the determinator 32 does not detect a defect related to stapling in step S15, the ejection mechanism 16 receives a control command from the system controller 27 and switches the conveyance destination of the bundle of sheets (step S19). Specifically, the ejection mechanism 16 switches the conveyance destination of the bundle of sheets from the non-defective product collecting section to the defective product collecting section by changing the posture from the first posture to the second posture. Thus, as illustrated in FIG. 1, a defective bundle 21 of sheets is collected by the defective product collecting section by falling by its own weight or the like. Therefore, even in a case where the determinator 32 detects a defective bundle of sheets, the inspection system 10 can continue production of booklets or the like without stopping the operation of the system.

Furthermore, when the processing in step S18 described above is completed, when the processing in step S19 described above is completed, or when the system controller 27 makes a negative determination in step S16 described above, the series of processing ends.

Although not illustrated in FIG. 1, the inspection system 10 may include a marking section instead of the ejection mechanism 16 or in addition to the ejection mechanism 16. The marking section applies marking to a bundle of sheets in accordance with results of the inspection by the determinator 32. Specifically, the marking section marks the bundle of sheets determined to be a defective product by the determinator 32. The marking method may be any method by which a person can visually identify whether the bundle of sheets is a non-defective product or a defective product. As a specific example of the marking method, a method for printing a mark on a bundle of sheets, a method for attaching a tag to a bundle of sheets, or the like is considered. In a case where the inspection system 10 includes the marking section, even when the determinator 32 detects a defective bundle of sheets, the inspection system 10 can continue production of a booklet or the like without stopping the operation of the system.

As described above, according to the inspection system 10 and the inspection method according to the embodiment of the present invention, it is possible to automatically inspect a bundle of sheets without manually performing the operation of inspecting the bundle of sheets. Thus, the process of producing a booklet can be automated.

Furthermore, since the acquirer 17 acquires three-dimensional information regarding a bundle of sheets, information regarding the shape of the bundle of sheets in a direction other than the surface direction, that is, in the height direction is obtained. Thus, the determinator 32 can detect defects that cannot be detected from a photograph or the like of a bundle of sheets taken from directly above. The above-described defects that cannot be detected from the photograph or the like include defects such as a fold and a tear of a sheet.

In addition, as the inspection of booklet binding, inspection of deformation of an outer shape of a booklet, such as a fold and a tear of the booklet, idle driving and buckling of staples, and the like are included. In a technology such as pattern matching that is a conventional method, a defective pattern to be detected needs to be manually defined. Therefore, it has been difficult for a technique such as pattern matching to cope with an undefined unknown defective pattern. Furthermore, in the conventional method for comparing an inspection image with a correct image, the allowable range for the ambiguity of input data is narrow, and it is difficult to set a threshold for a defect.

On the other hand, in the inspection system 10 according to the embodiment of the present invention, the determinator 32 inspects a bundle of sheets using the learning model 37 which is a deep learning model. For this reason, the determinator 32 can determine whether or not a product that is unclear as to whether the product is a non-defective is a non-defective product with a sense close to a human being. In particular, in digital printing, since a booklet can be produced from one volume, it is practically impossible to define a defective pattern each time as in the above-described pattern matching or the like. Therefore, it is highly effective to inspect a bundle of sheets using the deep learning model.

Furthermore, the acquirer 17 acquires the three-dimensional information of the bundle 21 of sheets on the downstream side of the post-processing machine 14, and the determinator 32 inspects the bundle 21 of sheets processed by the post-processing machine 14. Thus, the processing and the inspection of the bundle 21 of sheets are performed in-line. Therefore, the bundle 21 of sheets can be inspected without human intervention.

The post-processing machine 14 processes a plurality of sheets on which images have been printed by the printing machine 12 into a bundle of sheets. Thus, in addition to the above-described processing and the inspection of the bundle 21 of sheets, printing of an image is also performed in-line. Therefore, it is possible to suppress the occurrence of a mistake or waste due to the movement of the sheets from the printing machine 12 to the post-processing machine 14. Therefore, the inspection system 10 can increase reliability as a system, implement automation, and achieve labor saving.

The post-processing machine 14 is disposed downstream of the image inspection machine 13. Therefore, after an image of each of sheets forming a bundle of sheets is inspected by the image inspection machine 13, the processing by the post-processing machine 14 and the inspection of the bundle of sheets by the determinator 32 are performed. Thus, the entire bundle of sheets including the images of the respective sheets can be inspected.

### Modification Examples

However, the technical scope of the present invention is not limited to the above-described embodiment. The technical scope of the present invention also includes embodiments to which various modifications and improvements are added within a range in which specific effects obtained by the constituent features of the invention and combinations thereof can be obtained.

For example, in the above-described embodiment, the three-dimensional information has been described as an example of the information acquired by the acquirer 17, but the present invention is not limited thereto. The information acquired by the acquirer 17 may be two-dimensional information. Examples of the acquirer 17 that acquires two-dimensional information include a camera and a scanner. In addition, in a case where the information acquired by the acquirer 17 is two-dimensional information, the number of defect items of a bundle of sheets that can be detected by the determinator 32 can be increased by devising the arrangement of the camera that captures an image of the bundle of sheets. Specifically, the number of defect items of the bundle of sheets that can be detected by the determinator 32 can be increased by capturing an image of the bundle of sheets with the camera from an oblique direction. In addition, the number of defect items of the bundle of sheets that can be detected by the determinator 32 can be increased by capturing an image of the bundle of sheets from vertically above and from the horizontal direction using the cameras.

Furthermore, in the embodiment described above, when a bundle of sheets is inspected using the learning model 37 that is a deep learning model, the determinator 32 refers to a model corresponding to a combination of the basis weight of a cover sheet of the bundle of sheets and the number of sheets forming the bundle of sheets, but the present invention is not limited thereto. For example, the determinator 32 may refer to a model corresponding to the basis weight of the cover sheet of the bundle of sheets. In this case, a model trained for each basis weight of a cover sheet of a bundle of sheets may be stored in the learning database 42 as the deep learning model to be used to inspect the bundle of sheets. Further, the determinator 32 may refer to a model corresponding to the number of sheets forming the bundle of sheets. In this case, a model trained for each number of sheets forming a bundle of sheets may be stored in the learning database 42 as a deep learning model to be used for inspection of the bundle of sheets.

Some of the functions implemented by the inspection computer 18 may be provided in an external computer communicable with the inspection computer 18 via a network. For example, the learning database 42 that stores the learning model 37 may be included in the above-described external computer. The determinator 32 may inspect a bundle of sheets by exchanging data such as an image between the inspection computer 18 and an external computer.

Furthermore, the present invention is not limited to the inspection system and the inspection method, but can also be provided as an inspection program or a non-transitory recording medium storing the computer-readable inspection program. The inspection program is a program for causing a computer to execute acquiring information regarding a shape of a bundle including a plurality of processed recording media, and inspecting the bundle based on the acquired information and determining whether or not the bundle is a non-defective product. Further, the non-transitory recording medium storing the computer-readable program is, for example, a CD-ROM.

Although embodiments of the present invention have been described and illustrated in detail, the disclosed embodiments are made for purposes of illustration and example only and not limitation. The scope of the present invention should be interpreted by terms of the appended claims.

What is claimed is:

1. An inspection system that inspects a bundle including a plurality of processed recording media, the inspection system comprising:
an acquirer that acquires information regarding a shape of the bundle including the plurality of processed recording media; and
a hardware processor, wherein
the hardware processor inspects the bundle based on the information acquired by the acquirer.

2. The inspection system according to claim 1, wherein the hardware processor inspects the bundle using a deep learning model.

3. The inspection system according to claim 2, wherein the hardware processor uses a model of an unsupervised learning method as the deep learning model.

4. The inspection system according to claim 2, wherein the hardware processor uses an autoencoder as the deep learning model.

5. The inspection system according to claim 2, wherein
the deep learning model is a model trained for each basis weight of a cover sheet of the bundle, and
when the hardware processor inspects the bundle using the deep learning model, the hardware processor refers to a model corresponding to the basis weight of the cover sheet of the bundle.

6. The inspection system according to claim 2, wherein
the deep learning model is a model trained for each number of recording media forming the bundle, and
when the hardware processor inspects the bundle using the deep learning model, the hardware processor refers to a model corresponding to the number of recording media forming the bundle.

7. The inspection system according to claim 2, wherein
the deep learning model is a model trained for each combination of the basis weight of the cover sheet of the bundle and the number of recording media forming the bundle, and
when the hardware processor inspects the bundle using the deep learning model, the hardware processor refers to a model corresponding to the combination.

8. The inspection system according to claim 1, wherein the information acquired by the acquirer is three-dimensional information.

9. The inspection system according to claim 2, wherein
the information acquired by the acquirer is three-dimensional information,
the hardware processor normalizes the three-dimensional information according to a thickness and a size of the bundle, and
the deep learning model uses data normalized by the hardware processor.

10. The inspection system according to claim 2, wherein
the information acquired by the acquirer is three-dimensional information, and
the hardware processor generates an image of the three-dimensional information, and
data of the image generated by the hardware processor is used for the deep learning model.

11. The inspection system according to claim 8, wherein the three-dimensional information is represented by a two-dimensional heat map or two-dimensional contour lines.

12. The inspection system according to claim 1, wherein the information acquired by the acquirer is two-dimensional information.

13. The inspection system according to claim 1, further comprising a processing section that generates a bundle including a plurality of recording media by processing, wherein the hardware processor inspects the bundle processed by the processing section.

14. The inspection system according to claim 13, further comprising a printer that prints images on the recording media, wherein the plurality of recording media on which the images have been printed by the printer are processed by the processing section.

15. The inspection system according to claim 14, comprising an image inspector that inspects the images printed on the recording media by the printer, wherein the plurality of recording media on which the images have been inspected by the image inspector are processed by the processing section.

16. The inspection system according to claim 1, wherein the acquirer acquires the information using light emitted to the bundle.

17. The inspection system according to claim 1, wherein
the acquirer includes a first acquirer that acquires information regarding a shape of the bundle on a front side of the bundle, and a second acquirer that acquires information regarding a shape of the bundle on a back side of the bundle.

18. The inspection system according to claim 1, comprising an ejector that switches a conveyance destination of the bundle in accordance with a result of the inspection by the hardware processor.

19. The inspection system according to claim 1, further comprising a marking section that applies marking to the bundle in accordance with a result of the inspection by the hardware processor.

20. The inspection system according to claim 1, further comprising a system controller, wherein
the processing includes stapling, and
the system controller stops operation of the system when the hardware processor determines a defect related to the stapling.

21. The inspection system according to claim 1, wherein, when the hardware processor determines that the bundle to be inspected is a defective product, the hardware processor records information regarding the defective product.

22. An inspection method for inspecting a bundle including a plurality of processed recording media, the inspection method comprising:
acquiring information regarding a shape of the bundle including the plurality of processed recording media; and
inspecting the bundle based on the acquired information.

23. A non-transitory recording medium storing a computer-readable inspection program for inspecting a bundle including a plurality of processed recording media, the inspection program causing a computer to execute:
acquiring information regarding a shape of the bundle including the plurality of processed recording media; and
inspecting the bundle based on the acquired information and determining whether or not the bundle is a non-defective product.
